(19) United States
(12) Patent Application Publication
(10) Pub. No.: US 2026/0102618 A1
(43) Pub. Date: Apr. 16, 2026

(54) Implantable Stimulator Device With Controllable Bleed Resistors At All Electrodes

(71) Applicant: Boston Scientific Neuromodulation Corporation, Valencia, CA (US)

(72) Inventors: Kiran K. Gururaj, Valencia, CA (US); Philip L. Weiss, Sherman Oaks, CA (US); Goran N. Marnfeldt, Valencia, CA (US); Emanuel Feldman, Simi Valley, CA (US)

(21) Appl. No.: 19/299,186

(22) Filed: Aug. 13, 2025

Related U.S. Application Data
(60) Provisional application No. 63/705,630, filed on Oct. 10, 2024.

(51) Int. Cl. A61N 1/36; A61N 1/05; A61N 1/375

(52) U.S. Cl. CPC A61N 1/36125; A61N 1/0551; A61N 1/3756; A61N 1/36062

(57) ABSTRACT

Stimulator circuitry useable in a stimulator device is described. The stimulator circuitry provides individually controllable bleed resistors for each electrode, which are configurable to bleed residual charge from the electrodes' DC-blocking capacitor. The described circuitry is particularly useful for sensing electrical potentials in a patient's tissue, for example, evoked neural potentials. The programable bleed resistors address complications caused by DC-offset at the stimulator device's sensing amplifiers.

### CROSS REFERENCE TO RELATED APPLICATIONS

This is a non-provisional application of U.S. Provisional Patent Application Ser. No. 63/705,630, filed Oct. 10, 2024, which is incorporated herein by reference in its entirety, and to which priority is claimed.

### FIELD OF THE INVENTION

This application relates to Implantable Medical Devices (IMDs), and more specifically to implantable stimulator devices having controllable bleed resistors associated with each electrode.

### INTRODUCTION

Implantable neurostimulator devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Spinal Cord Stimulation (SCS) or Deep Brain Stimulation (DBS) system. However, the present invention may find applicability with any stimulator device system.

A stimulator system typically includes an Implantable Pulse Generator (IPG) 10 shown in FIG. 1. The IPG 10 includes a biocompatible device case 12 that holds the circuitry and a battery 14 for providing power for the IPG to function. The IPG 10 is coupled to tissue-stimulating electrodes 16 via one or more electrode leads that form an electrode array 17. For example, one or more percutaneous leads 15 can be used having ring-shaped or split-ring electrodes 16 carried on a flexible body 18. In another example, a paddle lead 19 provides electrodes 16 positioned on one of its generally flat surfaces. Lead wires 20 within the leads are coupled to the electrodes 16 and to proximal contacts 21 insertable into lead connectors 22 fixed in a header 23 on the IPG 10, which header can comprise an epoxy for example. Once inserted, the proximal contacts 21 connect to header contacts 24 within the lead connectors 22, which are in turn coupled by feedthrough pins 25 through a case feedthrough 26 to stimulation circuitry 28 within the case 12.

In the illustrated IPG 10, there are thirty-two electrodes (E1-E32), split between four percutaneous leads 15, or contained on a single paddle lead 19, and thus the header 23 may include a 2×2 array of eight-electrode lead connectors 22. However, the type and number of leads, and the number of electrodes, in an IPG is application specific and therefore can vary. The conductive case 12, or some conductive portion of the case, can also comprise an electrode (Ec). In an SCS application, the electrode lead(s) are typically implanted in the spinal column proximate to the dura in a patient's spinal cord, preferably spanning left and right of the patient's spinal column. The proximal contacts 21 are tunneled through the patient's tissue to a distant location such as the buttocks where the IPG case 12 is implanted, at which point they are coupled to the lead connectors 22. In a DBS application, the electrode leads are implanted in the brain through holes in the skull, and lead extensions are used to connect the leads to the IPG which is typically implanted under the clavicle (collarbone). In other IPG examples designed for implantation directly at a site requiring stimulation, the IPG can be lead-less, having electrodes 16 instead appearing on the body of the IPG 10 for contacting the patient's tissue. The IPG lead(s) can be integrated with and permanently connected to the IPG 10 in other solutions. SCS therapy can relieve symptoms such as chronic back pain, while DBS therapy can alleviate Parkinsonian symptoms such as tremor and rigidity.

IPG 10 can include an antenna 27a allowing it to communicate bi-directionally with a number of external devices discussed subsequently. Antenna 27a as shown comprises a conductive coil within the case 12, although the coil antenna 27a can also appear in the header 23. When antenna 27a is configured as a coil, communication with external devices preferably occurs using near-field magnetic induction. IPG 10 may also include a Radio-Frequency (RF) antenna 27b. In FIG. 1, RF antenna 27b is shown within the header 23, but it may also be within the case 12. RF antenna 27b may comprise a patch, slot, or wire, and may operate as a monopole or dipole. RF antenna 27b preferably communicates using far-field electromagnetic waves, and may operate in accordance with any number of known RF communication standards, such as Bluetooth, Zigbee, WiFi, MICS, and the like.

Stimulation in IPG 10 is typically provided by pulses each of which may include a number of phases (30i), as shown in the example of FIG. 2A. Stimulation parameters typically include amplitude (current I, although a voltage amplitude V can also be used); frequency (F); pulse width (PW); the electrodes 16 selected to provide the stimulation; and the polarity of such selected electrodes, i.e., whether they act as anodes that source current to the tissue or cathodes that sink current from the tissue. These and possibly other stimulation parameters taken together comprise a stimulation program that the stimulation circuitry 28 in the IPG 10 can execute to provide therapeutic stimulation to a patient.

In the example of FIG. 2A, electrode E1 has been selected as an anode (during its first phase 30a), and thus provides pulses which source a positive current of amplitude +I to the tissue. Electrode E2 has been selected as a cathode (again during first phase 30a), and thus provides pulses which sink a corresponding negative current of amplitude −I from the tissue. This is an example of bipolar stimulation, in which the lead includes one anode pole and one cathode pole. Note that more than one electrode on the lead may be selected to act as an anode electrode to form an anode pole at a given time, and more than one electrode may be selected to act as a cathode to form a cathode pole at a given time, as explained further in U.S. Pat. No. 10,881,859. Stimulation provided by the IPG 10 can also be monopolar, although this example is not yet shown. In monopolar stimulation, the lead is programmed with a single pole of a given polarity (e.g., a cathode pole), with the conductive case electrode Ec acting as a return (e.g., an anode pole). Again, more than one electrode on the lead may be active to form the pole during monopolar stimulation.

IPG 10 as mentioned includes stimulation circuitry 28 to form prescribed stimulation at a patient's tissue. FIG. 3 shows an example of stimulation circuitry 28, which includes one or more current source circuits and one or more current sink circuits. The sources and sinks constitute driver circuitry configured to drive current through the patient's tissue. The sources and sinks can comprise Digital-to-Analog converters (DACs), and may be referred to as PDACs and NDACs in accordance with the Positive (sourced, anodic) and Negative (sunk, cathodic) currents they respectively issue. In the example shown, a NDACi/PDACi pair is dedicated (hardwired) to a particular electrode node ei 39. Each electrode node ei 39 is connected to an electrode Ei 16 via a DC-blocking capacitor Ci 38, for the reasons explained below. The stimulation circuitry 28 in this example also supports selection of the conductive case 12 as an electrode (Ec 12), which case electrode is typically selected for monopolar stimulation as explained above. PDACs and NDACs can also comprise voltage sources.

Proper control of the PDACs and NDACs allows any of the electrodes 16 to act as anodes or cathodes to create a current through a patient's tissue, R, hopefully with good therapeutic effect. Consistent with the example provided in FIG. 2A, FIG. 3 shows operation during the first phase 30a in which electrode E1 has been selected as an anode electrode to source current I to the tissue R and E2 has been selected as a cathode electrode to sink current from the tissue. Thus, PDAC1 and NDAC2 are digitally programmed to produce the desired current, I, with the correct timing (e.g., in accordance with the prescribed frequency and pulse widths). As mentioned above, more than one anode electrode and more than one cathode electrode may be selected at one time, and thus current can flow through the tissue R between two or more of the electrodes 16.

Other stimulation circuitries 28 can also be used in the IPG 10. In an example not shown, a switching matrix can intervene between the one or more PDACs and the electrode nodes ei 39, and between the one or more NDACs and the electrode nodes. Switching matrices allows any PDAC or NDAC to be connected to any of the electrode nodes. Various examples of stimulation circuitries can be found in U.S. Pat. Nos. 6,181,969, 8,606,362, 8,620,436, 11,040,192, and 10,912,942. Much of the stimulation circuitry 28 of FIG. 3, including the PDACs and NDACs, the switch matrices (if present), and the electrode nodes ei 39 can be integrated on one or more Application Specific Integrated Circuits (ASICs), as described in U.S. Patent Application Publications 2012/0095529, 2012/0092031, and 2012/0095519. As explained in these references, ASIC(s) may also contain other circuitry useful in the IPG 10, such as IPG master control circuitry 102 (see FIG. 5), telemetry circuitry (for interfacing off chip with telemetry antennas 27a and/or 27b), circuitry for generating the compliance voltage VH (as explained next), various measurement circuits, etc.

Power for the stimulation circuitry 28 is provided by a compliance voltage VH, as described in further detail in U.S. Patent Application Publications 2013/0289665 and 2018/0071520. The compliance voltage VH may be coupled to the source circuitry (e.g., the PDAC(s)), while ground may be coupled to the sink circuitry (e.g., the NDAC(s)), such that the stimulation circuitry 28 is powered by VH and ground. Other power supply voltages may be used with the PDACs and NDACs, and explained in U.S. Patent Application Publication 2018/0071520, but these aren't shown in FIG. 3 for simplicity.

Preferably, and as described in U.S. Pat. No. 11,040,202, the compliance voltage VH can be produced by a VH regulator 49. VH regulator 49 receives the voltage of the battery 14 (Vbat) and boosts this voltage to a higher value required for the compliance voltage VH. VH regulator 49 can comprise an inductor-based boost converter or a capacitor-based charge pump for example. The regulator 49 can vary the value of VH based on measurements taken from the stimulation circuitry 28. As explained in detail in the '202 patent, VH measurement circuitry 51 can be used to measure the voltage drops across the active DACs (e.g., PDAC1 (Vp1) and NDAC2 (Vn2) in the example shown in FIG. 3) in the stimulation circuitry 28. Using such measurements allows VH to be established at an energy-efficient level: high enough to form the prescribed current without loading (i.e., without producing less current that prescribed), yet low enough to not needlessly waste power in the stimulation circuitry 28 when forming the prescribed current.

The VH measurement circuitry 51 can output an enable signal VH(en1) indicating when VH regulator 49 should increase the level of VH, i.e., when the voltage drops across the active DACs are too low. This enable signal VH(en1) may be processed at logic 53 in conjunction with other signals explained below to determine a master enable signal VH(en) for the VH regulator 49. Logic 53 may be associated with the IPG's control circuitry 102. Master enable signal VH(en) when asserted causes the VH regulator 49 to increase VH (e.g., when the current starts to load). Deasserting VH(en) disables the VH regulator, which allows VH to naturally decrease over time until it needs to be increased again. This feedback generally causes VH to be established at an energy-efficient value appropriate for the current that is being provided by the stimulation circuitry 28.

Also shown in FIG. 3 are DC-blocking capacitors Ci 38 placed in series in the electrode current paths between each of the electrode nodes ei 39 and the electrodes Ei 16 (including the case electrode Ec 12). The DC-blocking capacitors 38 act as a safety measure to prevent DC current injection into the patient, as could occur for example if there is a circuit fault in the stimulation circuitry 28. The DC-blocking capacitors 38 are typically provided off-chip (off of the ASIC(s)), and instead may be provided in or on a circuit board in the IPG 10 used to integrate its various components, as explained in U.S. Patent Application Publication 2015/0157861.

Referring again to FIG. 2A, the stimulation pulses as shown are biphasic, with each pulse comprising a first phase 30a followed thereafter by a second phase 30b of opposite polarity. Biphasic pulses are useful to actively recover any charge that might be stored on capacitive elements in the electrode current paths, such as on the DC-blocking capacitors 38. Charge recovery is shown with reference to both FIGS. 2A and 2B. During the first pulse phase 30a, charge will (primarily) build up across the DC-blockings capacitors C1 and C2 associated with the electrodes E1 and E2 used to produce the current, giving rise to voltages Vc1 and Vc2 (I=C * dV/dt). During the second pulse phase 30b, when the polarity of the current I is reversed at the selected electrodes E1 and E2, the stored charge on capacitors C1 and C2 is recovered, and thus voltages Vc1 and Vc2 hopefully return to 0V at the end the second pulse phase 30b. To recover all charge by the end of the second pulse phase 30b of each pulse (Vc1=Vc2=0V), the first and second phases 30a and 30b are charged balanced at each electrode, with the phases comprising an equal amount of charge but of the opposite polarity. In the example shown, such charge balancing is achieved by using the same pulse width (PWa=PWb) and the same amplitude (|+I|=|−I|) for each of the pulse phases 30a and 30b. However, the pulse phases 30a and 30b may also be charged balance if the product of the amplitude and pulse widths of the two phases 30a and 30b are equal, as is known.

Charge recovery using phases 30a and 30b is said to be “active” because the P/NDACs in stimulation circuitry 28 actively drive a current, in particular during the last phase 30b to recover charge stored after the first phase 30a. However, such active charge recovery may not be perfect, and some residual charge may be present in capacitive structures even after phase 30b is completed. Accordingly, the stimulation circuitry 28 can also provide for passive charge recovery. Passive charge recovery is implemented using passive charge recovery switches PRi 41 as shown in FIG. 3. These switches 41 when selected via assertion of control signals <Xi> couple each electrode node ei to a passive recovery voltage Vpr established on bus 43. As explained in U.S. Pat. Nos. 10,716,937 and 10,792,491, this allows any stored charge to be passively recovered through the patient's tissue, R, without actively driving currents using the P/NDACs. Control signals <Xi> are usually asserted to cause passive charge recovery after each pulse (e.g., after each last phase 30b) during periods 30c shown in FIG. 2A. Because passive charge recovery involves capacitive discharge through the resistance R of the patient's tissue, such discharge manifests as an exponential decay in current, as shown in FIG. 2A.

As also discussed in the '937 patent, each of the passive charge recovery switches 41 can be associated with a variable resistance, and as such each switch 41 can be controlled by a bus of signals <Xi> to control the resistance at which passive charge recovery occurs—i.e., the on resistance of the switches 41 when they are closed. Note that the common voltage Vpr used during passive charge recovery can comprise ground, VH, VH/2, the voltage of the battery 14 (Vbat), or any other DC voltage provided by the IPG 10, and any number of generator circuits (not shown) can be used to produce these voltages for Vpr. Passive charge recovery during period 30c may be followed by a quiet period 30d during which no active current is driven by the DAC circuitry, and none of the passive recovery switches 41 are closed. This quiet period 30d may last until the next pulse is actively produced (e.g., phase 30a). Like the particulars of pulse phases 30a and 30b, the occurrence of passive charge recovery (30c) and any quiet periods (30d) can be prescribed as part of the stimulation program.

Although not illustrated, the pulses provided by the IPG 10 may also be single-phase (i.e., monophasic) pulses having a single polarity, and thus may lack a second (opposite polarity) pulse phase that provides active charge recovery. Performing passive charge recovery can be more important when monophasic pulses are used, and indeed may be required, as discussed further later.

FIG. 4 shows various external systems 60, 70, and 80 that can wirelessly communicate data with the IPG 10 (which again can include an ETS). Such systems can be used to wirelessly transmit a stimulation program to the IPG 10—that is, to program its stimulation circuitry 28 to produce stimulation with desired amplitudes and timings as described earlier. Such systems may also be used to adjust one or more stimulation parameters of a stimulation program that the IPG 10 is currently executing, and/or to wirelessly receive information from the IPG 10, such as various status information, etc.

External controller 60 can be as described in U.S. Patent Application Publication 2015/0080982 for example, and may comprise a portable, hand-held controller dedicated to work with the IPG 10. External controller 60 may also comprise a general-purpose mobile electronics device such as a mobile phone which has been programmed with a Medical Device Application (MDA) allowing it to work as a wireless controller for the IPG 10, as described in U.S. Patent Application Publication 2015/0231402. External controller 60 includes a display 61 and a means for entering commands, such as buttons 62 or selectable graphical icons provided on the display 61. The external controller 60's user interface enables a patient to adjust stimulation parameters, although it may have limited functionality when compared to systems 70 and 80, described shortly. The external controller 60 can have one or more antennas capable of communicating with the IPG 10. For example, the external controller 60 can have a near-field magnetic-induction coil antenna 64a capable of wirelessly communicating with the coil antenna 27a in the IPG 10. The external controller 60 can also have a far-field RF antenna 64b capable of wirelessly communicating with the RF antenna 27b in the IPG 10.

Clinician programmer 70 is described further in U.S. Patent Application Publication 2015/0360038, and can comprise a computing device such as a desktop, laptop, or notebook computer, a tablet, a mobile smart phone, a Personal Data Assistant (PDA)-type mobile computing device, etc. In FIG. 4, the computing device is shown as a laptop computer that includes typical computer user interface means such as a display 71, buttons 72, as well as other user-interface devices such as a mouse, a keyboard, speakers, a stylus, a printer, etc., not all of which are shown for convenience. Also shown in FIG. 4 are accessory devices for the clinician programmer 70 that are usually specific to its operation as a stimulation controller, such as a communication “wand” 76 coupleable to suitable ports on the computing device. The antenna used in the clinician programmer 70 to communicate with the IPG 10 can depend on the type of antennas included in the IPG 10. If the patient's IPG 10 includes a coil antenna 27a, wand 76 can likewise include a coil antenna 74a to establish near-field magnetic-induction communications at small distances. In this instance, the wand 76 may be affixed in close proximity to the patient, such as by placing the wand 76 in a belt or holster wearable by the patient and proximate to the patient's IPG 10. If the IPG 10 includes an RF antenna 27b, the wand 76, the computing device, or both, can likewise include an RF antenna 74b to establish communication with the IPG 10 at larger distances. The clinician programmer 70 can also communicate with other devices and networks, such as the Internet, either wirelessly or via a wired link provided at an Ethernet or network port.

External system 80 comprises another means of communicating with and controlling the IPG 10 via a network 85 which can include the Internet. The network 85 can include a server 86 programmed with communication and control functionality, and may include other communication networks or links such as WiFi, cellular or land-line phone links, etc. The network 85 ultimately connects to an intermediary device 82 having antennas suitable for communication with the IPG's antenna, such as a near-field magnetic-induction coil antenna 84a and/or a far-field RF antenna 84b. Intermediary device 82 may be located generally proximate to the IPG 10. Network 85 can be accessed by any user terminal 87, which typically comprises a computer device associated with a display 88. External system 80 allows a remote user at terminal 87 to communicate with and control the IPG 10 via the intermediary device 82.

FIG. 4 also shows circuitry 90 involved in any of external systems 60, 70, or 80. Such circuitry can include control circuitry 92, which can comprise any number of devices such as one or more microprocessors, microcomputers, FPGAs, DSPs, other digital logic structures, etc., which are capable of executing programs in a computing device. Such control circuitry 92 may contain or coupled with memory 94 which can store external system software 96 for controlling and communicating with the IPG 10, and for rendering a Graphical User Interface (GUI) 99 on a display (61, 71, 88) associated with the external system. In external system 80, the external system software 96 would likely reside in the server 86, while the control circuitry 92 could be present in either or both the server 86 or the terminal 87.

### SUMMARY

Disclosed herein are stimulator devices configured to provide electrical stimulation, comprising: a plurality of electrode nodes, wherein each electrode node is couplable to a different electrode, wherein each electrode is configured to contact a patient's tissue, driver circuitry configured to drive at least two of the electrode nodes to provide current through the tissue, a plurality of DC-blocking capacitors, wherein each of the DC-blocking capacitors is connected in series between one of the electrode nodes and a different one of the electrodes, a plurality of first switches and a plurality of first resistors, wherein each of the plurality of first switches is serially connected to a different one of the plurality of first resistors, wherein each of the serially connected first switch and first resistor is coupled at one end to a different one of the plurality of electrode nodes and at a second end to a first bus, a plurality of second switches and a plurality of second resistors, wherein each of the plurality of second switches is serially connected to a different one of the plurality of second resistors, wherein each of the serially connected second switch and second resistor is individually controllable and is coupled at one end to a different one of the plurality of electrode nodes and at a second end to a second bus, control circuitry configured to: close at least one selected first switch to discharge charge on at least one of the DC-blocking capacitors to the first bus, and close at least one selected second switch to discharge charge on at least one of the DC-blocking capacitors to the second bus. According to some embodiments, the second bus comprises a third resistor in parallel with one of the DC-blocking capacitors and wherein the control circuitry is configured to selectively close a third switch to enable charge on the second bus to discharge through the third resistor. According to some embodiments, the stimulator device comprises a case that is configurable as a case electrode, wherein the third resistor is in parallel with the DC-blocking capacitor of the case electrode, and wherein charge discharged through the third resistor is discharged to the tissue via the case electrode. According to some embodiments, the stimulator devices comprise a plurality of fourth switches wherein each of fourth switches is individually coupled at one end to a different one of the plurality of electrode nodes and at a second end to a third bus, wherein the third bus is connected to a reference voltage source that is configured to provide a reference voltage. According to some embodiments, the control circuitry is configured to close one or more of the fourth plurality of switches to provide the reference voltage to a selected one or more of the electrode nodes. According to some embodiments, the driver circuitry is powered by a compliance voltage (VH) and wherein the reference voltage is a function of VH. According to some embodiments, the reference voltage is VH/2. According to some embodiments, the stimulator devices comprise a battery, wherein the reference voltage is a battery voltage or ground. According to some embodiments, the control circuitry is configured to selectively connect the second bus to the third bus. According to some embodiments, the stimulator devices comprise a plurality of fifth switches, wherein each of fifth switches is individually coupled at one end to a different one of the plurality of electrode nodes and at a second end to a fourth bus, wherein the fourth bus is connected to sensing circuitry. According to some embodiments, the control circuitry is configured to: select one or more of the plurality of electrode nodes as sensing electrode nodes, during a first phase, close the second switch of each of the sensing electrode nodes to bleed residual charge from the sensing electrode node's DC-blocking capacitor to the second bus, during a second phase, open the second switch of each of the sensing electrode nodes and close the fifth switch of each of the sensing electrode nodes. According to some embodiments, the sensing circuitry comprises a plurality of sensing amplifiers and wherein closing the fifth switch of each of the sensing electrode nodes comprises asserting sensing enable signals to each of the fifth switches, wherein the each of the sensing enable signals control the respective fifth switches to connect each of the sensing electrode nodes to selected sensing amplifiers of the plurality of sensing amplifiers. According to some embodiments, the control circuitry is configured to: select at least two of the electrode nodes as stimulating electrode nodes, during a first phase, cause the driver circuitry to drive the selected at least two stimulating electrode nodes to provide current through the tissue, during a second phase, use passive charge recovery to recover at least a portion of charge from the DC-blocking capacitors connected to the at least two stimulating electrode nodes, and during a third phase, bleeding residual charge from the DC-blocking capacitors connected to the at least two stimulating electrode nodes, wherein the residual charge comprises charge on the DC-blocking capacitors that was not recovered during the second phase. According to some embodiments, the control circuitry is configured to: open the first and second switches connected to the at least two stimulating electrode nodes during the first phase, close the first switches connected to the at least two stimulating electrode nodes during the second phase, and open the first switches and close the second switches connected to the at least two stimulating electrode nodes during the third phase. According to some embodiments, during the third phase, the control circuitry is configured to close a third switch, causing the residual charge to bleed to a selected electrode through a third resistor that is parallel across the selected electrode's DC-blocking capacitor. According to some embodiments, the selected electrode is a case electrode.

Also disclosed herein are stimulator devices configured to provide electrical stimulation, comprising: a plurality of electrode nodes, wherein each electrode node is couplable to a different electrode configured to contact a patient's tissue, wherein each electrode node may be configured as a stimulating electrode node to provide active stimulation or as sensing electrode node to sense electric potentials present at the electrode node, a plurality of DC-blocking capacitors, wherein each of the DC-blocking capacitors is connected in series between one of the electrode nodes and a different one of the electrodes, a plurality of passive charge recovery resistors, wherein each of the passive charge recovery resistors is coupled to a different one of the plurality of electrode nodes to passively recover charge from the node's DC-blocking capacitor, a plurality of individually controllable bleed resistors, wherein each of the bleed resistors is connected to a different one of the plurality of electrode nodes to bleed residual charge from the node's DC-blocking capacitor, and control circuitry configured to: selectively activate a first one or more of the bleed resistors and not activate a second one or more of the bleed resistors. According to some embodiments, the control circuitry is configured to: select a two or more of the plurality of electrode nodes as stimulating electrode nodes, select one or more of the plurality of electrode nodes as sensing electrode nodes, during a first phase, cause the two or more stimulating electrode nodes to deliver active stimulation, during a second phase, use the passive charge recovery resistors for the stimulating electrode nodes to passively recover charge from the stimulating electrode nodes' DC-blocking capacitors, during a third phase, use the one or more sensing electrode nodes to sense electric potentials present at the one or more sensing electrode nodes, during one or more of the first and second phases, activate the sensing electrode nodes' bleed resistors to bleed residual charge from the sensing electrode nodes' DC-blocking capacitors while not activating the stimulating nodes' bleed resistors. According to some embodiments, the control circuitry is configured to: during the third phase, activating the stimulating nodes' bleed resistors to bleed residual charge from the stimulating nodes' DC-blocking capacitors while not activating the sensing nodes' bleed resistors. According to some embodiments, each of the plurality of passive charge recovery resistors are variable resistors and wherein each of the plurality of bleed resistors are not variable resistors.

### BRIEF DESCRIPTION OF THE DRAWINGS

FIG. 1 shows an Implantable Pulse Generator (IPG), in accordance with the prior art.

FIGS. 2A and 2B show an example of stimulation pulses producible by the IPG, in accordance with the prior art.

FIG. 3 shows stimulation circuitry useable in the IPG, in accordance with the prior art.

FIG. 4 shows various external devices capable of communicating with and programming stimulation in an IPG, in accordance with the prior art.

FIG. 5 shows an improved IPG having neural response sensing capability.

FIG. 6 shows stimulation producing a neural response, and the sensing of that neural response at least one electrode of the IPG.

FIG. 7 shows biasing circuitry useable to set a common mode voltage Vcm to the patient's tissue.

FIG. 8 illustrates DC offset on a sense amplifier when sensing tissue potentials.

FIG. 9A illustrates an embodiment of IPG circuitry comprising individually controllable bleed resistors associated with each electrode node and FIG. 9B illustrates a variable resistor passive charge recovery network.

FIGS. 10A and 10B illustrate instantiation of IPG circuit components during stimulation and sensing.

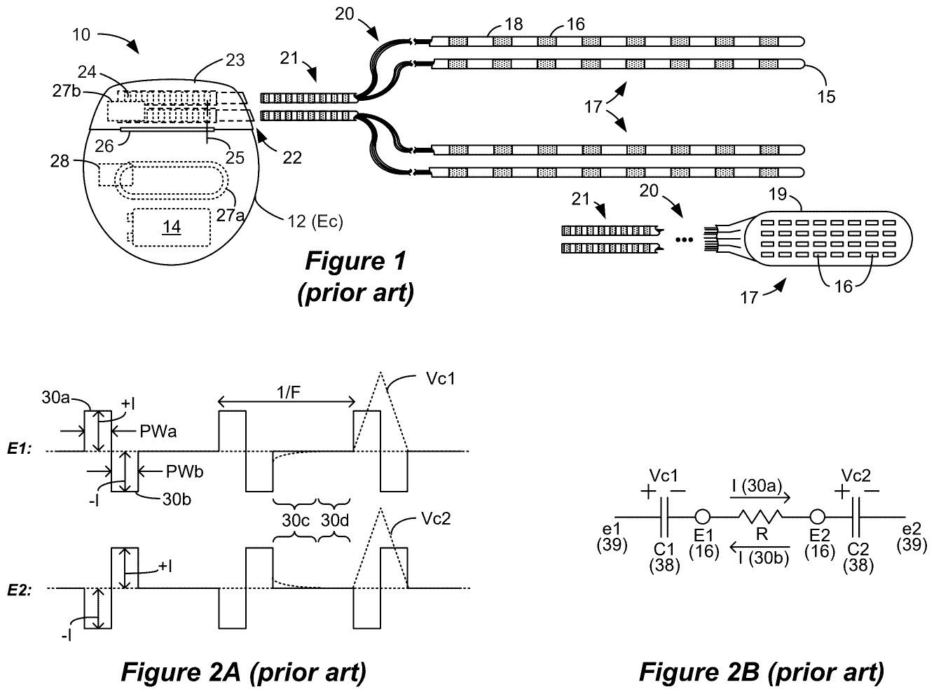

### DETAILED DESCRIPTION

An increasingly interesting development in pulse generator systems is the addition of sensing capability to complement the stimulation that such systems provide. For example, and as explained in U.S. Patent Application Publication 2017/0296823, it can be beneficial to sense a neural response produced by neural tissue that has received stimulation from an IPG. U.S. Patent Application Publication 2017/0296823 shows an example where sensing of neural responses is useful in an SCS context, and in particular discusses the sensing of Evoked Compound Action Potentials, or “ECAPs.” U.S. Patent Application Publication 2022/0040486 shows an example where sensing of neural responses is useful in a DBS context, and in particular discusses the sensing of Evoked Resonant Neural Activity, or “ERNA.”

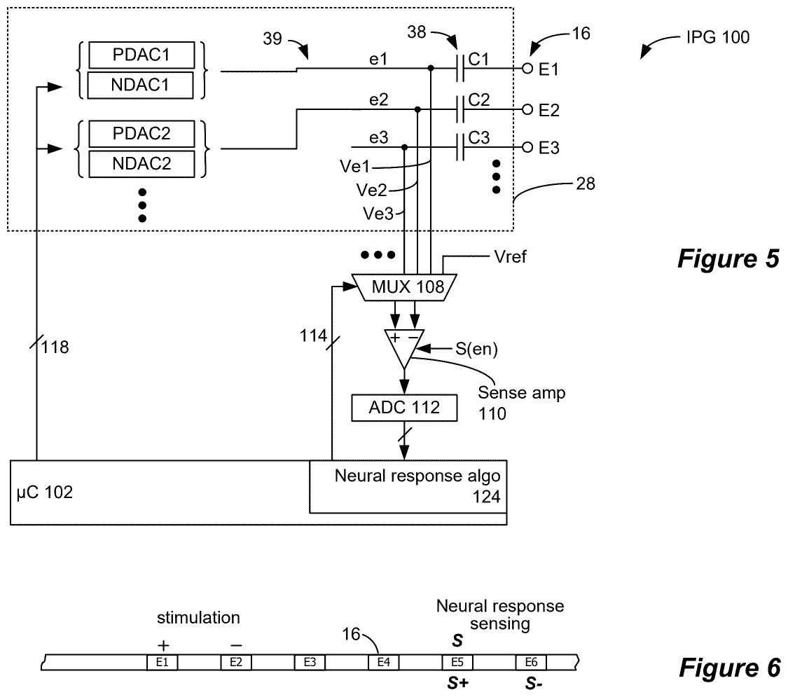

FIG. 5 shows circuitry for sensing neural responses in an IPG 10. The IPG 10 includes control circuitry 102, which may comprise a microcontroller for example, such as Part Number MSP430, manufactured by Texas Instruments, which is described in data sheets accessible on the Internet. Other types of control circuitry may be used in lieu of a microcontroller as well, such as microprocessors, FPGAs, DSPs, or combinations of these, etc. Control circuitry 102 may also be formed in whole or in part in one or more Application Specific Integrated Circuits (ASICs) in the IPG 10 as described earlier, which ASIC(s) may additionally include the other circuitry shown in FIG. 5.

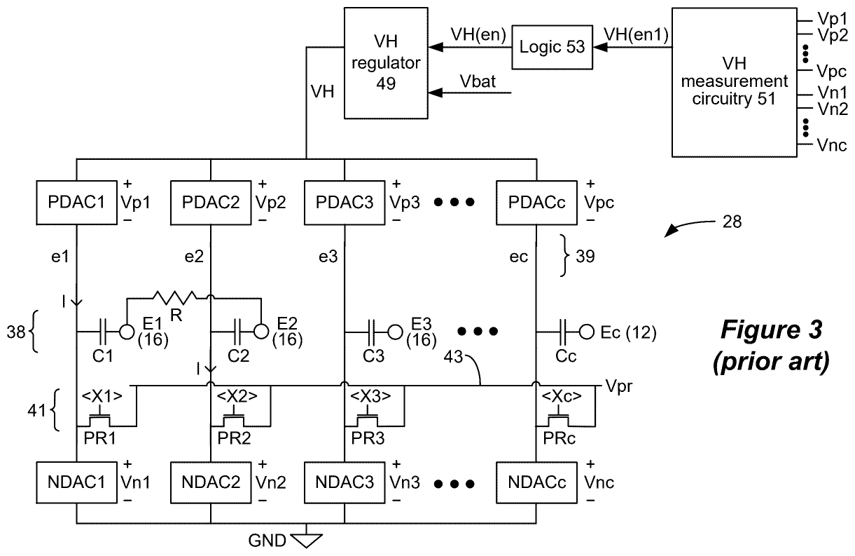

FIG. 5 includes the stimulation circuitry 28 described earlier (FIG. 3), including one or more DACs (PDACs and NDACs). A bus 118 provides digital control signals to the DACs to produce currents or voltages of prescribed amplitudes and with the correct timing at the electrodes selected for stimulation. The electrode current paths to the electrodes 16 include the DC-blocking capacitors 38 described earlier.

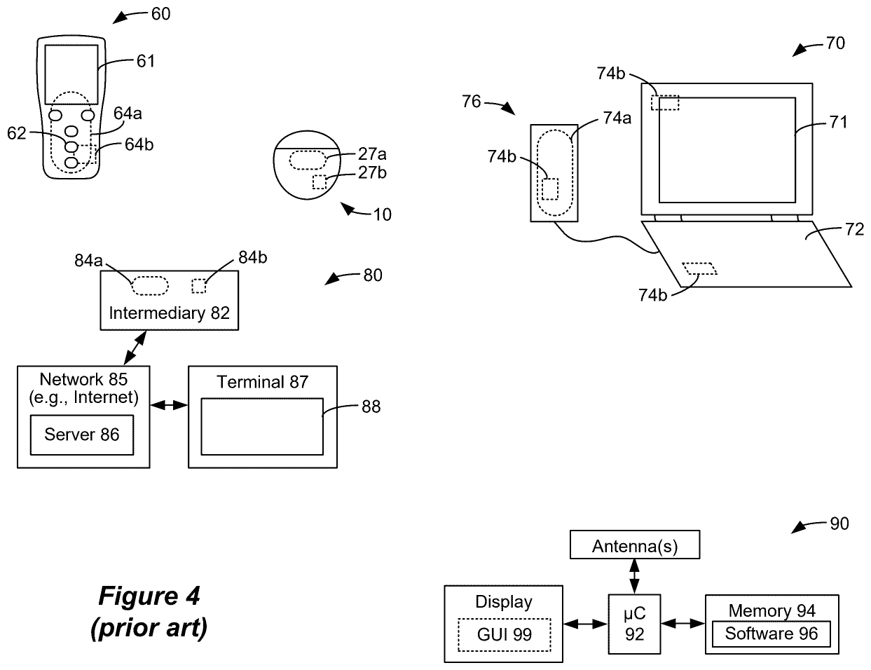

FIG. 5 also shows circuitry used to detect neural responses. As shown, the electrode nodes 39 are input to a multiplexer (MUX) 108. The MUX 108 is controlled by a bus 114, which operates to select one or more electrode nodes, and hence to designate corresponding electrodes 16 as sensing electrodes. The sensing electrode(s) selected via bus 114 can be determined automatically by control circuitry 102 and/or a neural response algorithm 124, as described further below. However, the sensing electrode(s) may also be selected by the user (e.g., a clinician) via an external system (FIG. 4).

Electrodes selected as sensing electrodes are provided by the MUX 108 to neural response detection circuitry. This circuitry can comprise a sense amplifier 110, and sensing can occur differentially using two sensing electrodes, or using a single sensing electrode. This is shown in the example of FIG. 6. If single-ended sensing is used, a single electrode (e.g., E5) is selected as a sensing electrode(S) and is provided to the positive terminal of the sense amp 110, where it is compared to a reference voltage Vref provided to the negative input. The reference voltage Vref can comprise any DC voltage produced within the IPG, such as ground. If differential sensing is used, two electrodes (e.g., E5 and E6) are selected as sensing electrodes (S+ and S−) by the MUX 108, with one electrode (e.g., E5) provided to the positive terminal of the sense amp 110, and the other (e.g., E6) provided to the negative terminal. Differential sensing can be useful to cancel any common mode voltages present in the tissue and reflected at the electrodes, such as voltages created by the stimulation itself. See, e.g., U.S. Patent Application Publication 2021/0236829. Although only one sense amp 110 is shown in FIG. 5 for simplicity, there could be more than one, such as a sense amp dedicated to each electrode node. In this case, MUX 108 would not be necessary, and each sense amp could be activated as needed depending on which electrodes are selected as sensing electrodes. The timing at which sensing occurs can be affected by a sensing enable signal S(en), as discussed further below.

The analog waveform comprising the sensed neural response and output by the sense amp 110 is preferably converted to digital signals by an Analog-to-Digital converter (ADC) 112, and input to the IPG's control circuitry 102. The ADC 112 can be included within the control circuitry 102's input stage as well. The control circuitry 102 can be programmed with a neural response algorithm 124 to evaluate the neural response, and to take appropriate actions as a result. For example, the neural response algorithm 124 may change the stimulation in accordance with the sensed neural response and can issue new control signals via bus 118 to change operation of the stimulation circuitry 28 to affect better treatment for the patient. The neural response algorithm 124 may also cause the selection of new sensing electrode(s), which can be affected by issuing new control signals on bus 114. Selecting optimal sensing electrode(s) can be important and may be determined in light of stimulation that is being provided. In this regard, sensing electrodes may be selected near enough to the electrodes providing stimulation (e.g., E1 and E2) to allow for proper neural response sensing, but far enough from the stimulation that the stimulation doesn't substantially interfere with neural response sensing. See, e.g., U.S. Patent Application Publication 2020/0155019.

Neural responses to stimulation are typically small-amplitude signals on the order of microVolts or milliVolts, which can make sensing difficult. The sense amp 110 needs to be capable of resolving this small signal, and this is particularly difficult when one realizes that this small signal typically rides on a background voltage otherwise present in the tissue. As explained in U.S. Pat. No. 11,040,202, which is incorporated by reference in its entirety, this background voltage can vary on the order of Volts, and can be caused by the stimulation itself. It is difficult to design sense amplifier circuitry 110 to reliably perform the task of accurately sensing a small-signal neural response while rejecting the background tissue voltage. Because stimulation causes the background tissue voltage to vary, it is preferred that neural responses are sensed after active stimulation is provided. Thus, sensing enable signal S(en) is preferably asserted during these times. That being said, stimulation artifacts resulting from the stimulation may still be present and cause variations in the tissue voltage even after stimulation has ceased. See, e.g., PCT (Int'l) Patent Application Publication WO 2020/251899.

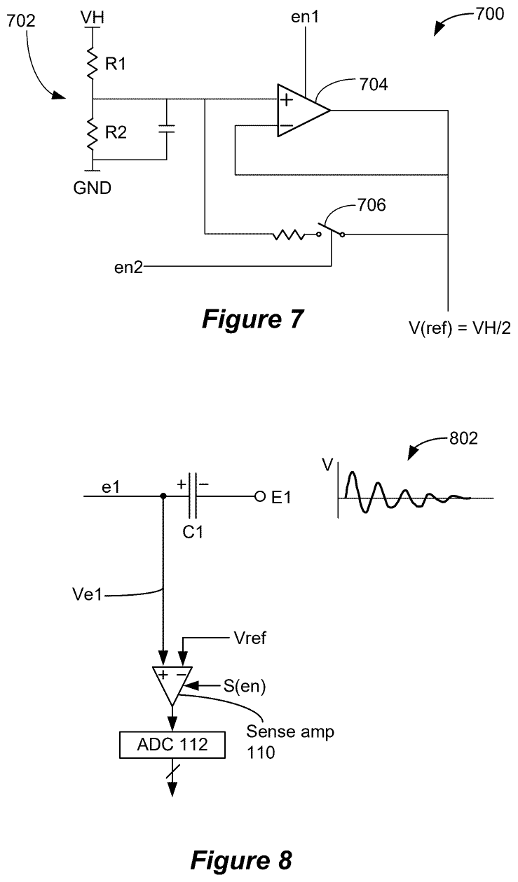

One way of addressing this issue of background tissue voltage variability is to drive and hold the tissue to a pseudo-constant common mode voltage (Vcm). The above-incorporated '202 patent and U.S. Patent Publication No. 2023/0138443, which is incorporated herein by reference, each describe circuitry to hold the tissue to a pseudo-constant programmable common mode voltage (Vcm). FIG. 7 illustrates another circuit for providing a reference voltage V(ref) that may be used to drive the tissue to a common mode voltage V(cm). The illustrated reference voltage circuit 700 provides a reference voltage of VH/2. The circuit 700 comprises a voltage divider 702 comprising resistors R1 and R2 to divide the compliance voltage VH. R1 and R2 may be equal to VH/2. R1 and R2 may each be 10 MΩ, for example. An operational amplifier 704 may be configured as a voltage follower and enabled by assertion of an enable signal en1. The circuit may also include a switch 706 which may be enabled by assertion of an enable signal en2. According to some embodiments, the enable signal en1 may be selected to provide the reference voltage for low impedance loads and the enable signal en2 may be selected to provide the reference voltage for high impedance loads.

As explained above, charge may build up across an electrode node's blocking capacitor when the electrode node is activated to deliver stimulation to the patient. As also explained above, active and/or passive charge recovery may be used to discharge the blocking capacitors. However, active and/or passive charge recovery may not completely remove all the charge on the capacitor, which may result in residual charge accumulation at the capacitors. Such accumulation of residual charge can be detrimental to the ability to reliably sense small signals at effected electrodes. For example, FIG. 8 shows a simplified schematic of a portion of the sensing circuitry described above (FIG. 5) with portions removed for clarity. Assume that a neural response 802 is being recorded at a sensing electrode E1 and that the neural response is being recorded with respect to a reference voltage Vref. The voltage Ve1, which represents the voltage measured in the tissue, is provided to the sense amplifier 110. But if residual charge has accumulated on the blocking capacitor C1, Ve1 also contains DC voltage due to that charge. Thus, there is a DC offset between the two inputs of the sense amplifier that can confound the measurement of the tissue voltage (i.e., the neural response), especially when the tissue voltage has a small amplitude.

Embodiments of the disclosed IPGs address the problem of accumulated residual charge by providing bleed resistors for each of the electrode nodes that are configured to bleed the residual charge from each of the blocking capacitors. Each of the bleed resistors is independently controllable (i.e., independently addressable and programmable), providing flexibility for a variety of stimulation and sensing configurations. Each of the bleed resistors may be associated with its own switch, such that when the switch is closed, the bleed resistor provides a DC path for residual charge stored on the respective associated DC-blocking capacitor, and when the switch is open, the associated bleed resistor is open circuited (i.e., no DC path through that bleed resistor). Saying that each of the bleed resistors is independently controllable means that they can be activated independently. For example, the bleed resistors for some of the electrode nodes may be activated while others are open circuited. According to some embodiments, a sub-set (or all) of the bleed resistor switches may be ganged together to open or close the DC paths for all of the ganged electrode nodes at once.

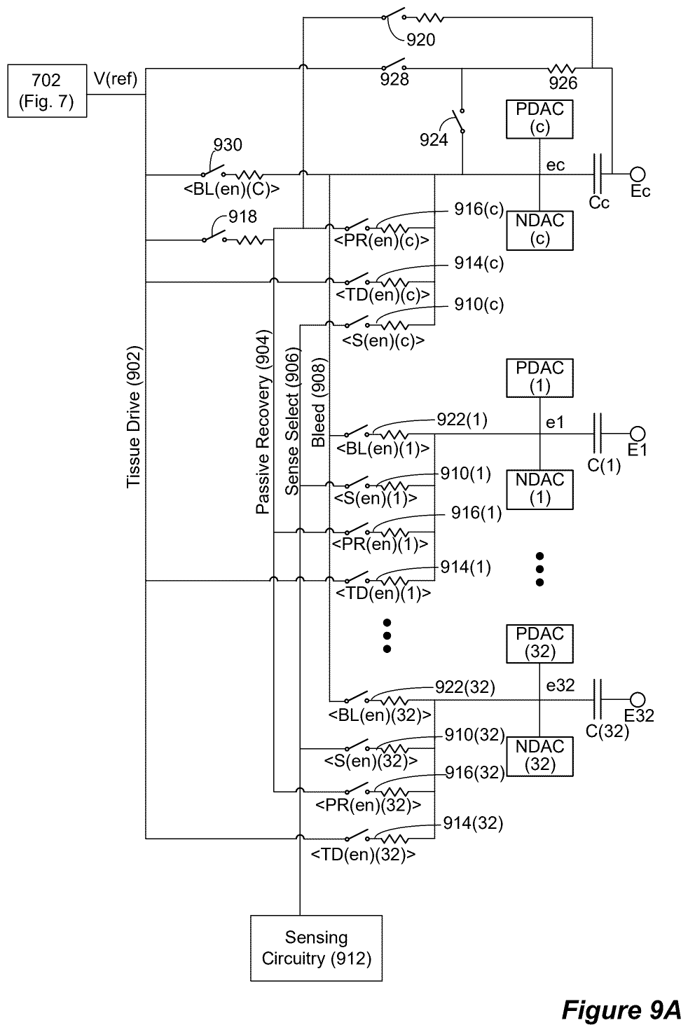

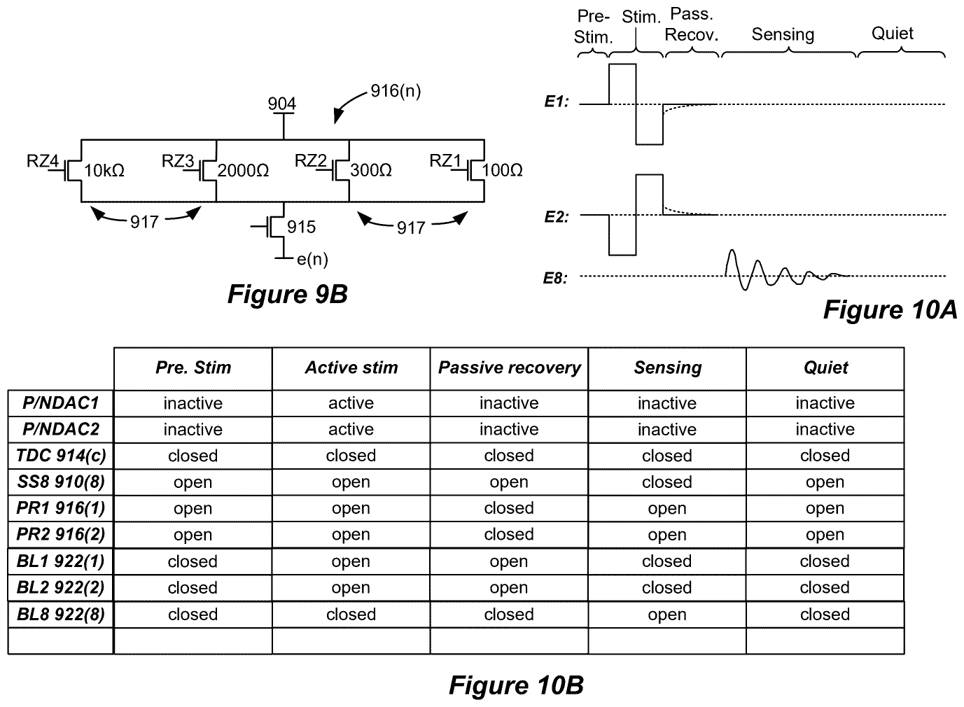

FIG. 9 illustrates aspects of IPG circuitry 900 for providing stimulation and sensing of tissue electrical signals. The illustrated IPG circuitry is configured for 32 lead-based electrodes and a case electrode. The electrodes are denoted as Ec (i.e., case electrode) and E1-E32. Each electrode is associated with PDACs and NDACs, electrode nodes ec-e32, and blocking capacitors C(c)-C32, which are as described above.

Four buses are illustrated in the embodiment shown in FIG. 9. The buses are introduced here, and specific implementations of the buses are described in more detail below. Bus 902 is referred to as a tissue drive bus. The bus 902 is connected to a reference voltage (V(ref)) source. The source of V(ref) may be a circuit, such as circuit 702 (FIG. 7). One of the uses of the bus 902 is to drive the patient's tissue to a common mode reference voltage, as described above. Bus 904 is referred to as the passive recovery bus and is implemented in passive charge recovery, which is described above. Bus 906 is referred to as a sense select bus and is used to instantiate given electrodes for sensing/recording. Bus 908 is referred to as a bleed bus and is used to provide a DC path to bleed accumulated residual charge on the blocking capacitors.

As with IPG circuitry discussed above (FIG. 3), the PDACs/NDACs of any of the electrode nodes ec-e32 of the IPG circuitry 900 may be digitally programmed to provide current to their respective electrodes. Control and power circuitry for the PDACs/NDACs is omitted from FIG. 9 for the sake of clarity, but the reader is referred to FIG. 3, for example.

Any of the electrode nodes/electrodes of the IPG circuitry 900 may also be enabled for sensing/recording electrical potentials present in the patient's tissue. In the illustrated embodiment, sense select switches 910(c)-910(32) are associated with each of the electrode nodes ec-e32, respectively. The sense select switches may be enabled by a sense enable signal <S(en)(n)> to connect the respective electrode node to sensing circuitry 912 via the sense select bus 906. According to some embodiments, the sensing circuitry 912 may comprise a plurality of sense amplifiers. For example, U.S. Non-Provisional Ser. No. 19/055,361 , filed Feb. 17, 2025, the entire contents of which are incorporated herein by reference, describes sensing circuitry including a downselector with switching networks used to couple selected electrode nodes and/or DC voltages to one or more selected sense amplifiers. The downselector can be controlled to couple one or more electrodes nodes to a particular sense amp during sensing durations. According to some embodiments, the enablement signal <S(en)(n)> may comprise multiple bits of information configured to control the switching network/downselector.

As mentioned above, the embodiments of the IPG circuitry 900 are configured to drive the tissue to a programmable common mode reference voltage V(ref), e.g., VH/2, VH, Vbat, ground, or the like. Accordingly, each electrode node ec-e32 is configured with a tissue drive switch 914(c)-914(32), respectively. Each of the respective tissue drive switches may be enabled by an tissue drive enable signal <TD(n)> that may be asserted to connect the respective electrode node e(n) to the tissue drive bus 902.

Each electrode node ec-e32 is also configured with a passive charge recovery switch 916(c)-916(32) that may be enabled to effect passive charge recovery, as described above. Each of the passive charge recovery switches may be enabled by a passive charge recovery enable signal <PR(en)(n)>, which may be asserted to connect the respective electrode node to the passive recovery bus 904. According to some embodiments, each of the passive charge recovery switches may comprise variable resistors (or a plurality of switches) configured to provide a variable resistance for passive charge recovery. For example, U.S. Pat. No. 10,716,937 (“the '937 Patent”), the entire contents of which are incorporated herein by reference, describes such a network for providing variable and programmable resistance during passive charge recovery. FIG. 9B illustrates an embodiment of such a passive charge recovery switch 916(n), as described in the '937 Patent, connected between an electrode node e(n) and the passive charge recovery bus 904. The passive charge recovery switch 916(n) comprises a first switch 915 serially connected to a network of resistance transistor/switches 917 that are in parallel with each other. Resistance control signals RZ[4:1] are each received at the gate of one of the resistance transistor/switches 917. Each of resistance transistors 917 is preferably sized differently to provide a different resistance. In the example shown, RZ1 controls a resistance transistor 917 of 100 ohms; RZ2 controls 300 ohms; RX3 controls 2000 ohms; and RZ4 controls 10000 ohms. Accordingly, the passive charge recovery enable signal <PR(en)(n)> may comprise the bits of information configured to select the resistance of the respective passive charge recovery switch 916(n). It should be noted that the resistance transistor/switches 917 are referred to herein as “resistors” even though they are implemented as transistor/switches in some embodiments.

The illustrated IPG circuitry 900 provides two modes for passive charge recovery. In a first mode, charge stored on the blocking capacitors C(n) can be recovered during passive charge recovery by connecting the passive charge recovery bus 904 to the tissue drive bus 902 via the switch 918. The switch 918 may be associated with a resistor, as shown, which may have a large value, for example, about 5 MΩ. Thus, the charge on the blocking capacitors C(n) may be recovered to V(ref). In a second mode, the charge on the blocking capacitors C(n) may be provided to the case electrode Ec via a switch 920, where the charge may be dissipated through the tissue impedance. The switch 920 may also be associated with a resistor, which according to some embodiments, may be a variable resistor. This is particularly useful for removing DC bias on the case blocking capacitor C(c). In the rest of this disclosure, the two various modes or passive charge recovery will not be distinguished.

As mentioned above, passive charge recovery may not completely discharge accumulated charge on the blocking capacitors. Thus, the embodiments of the IPG circuitry 900 include various bleed resistors and DC paths configured to bleed residual charge off the blocking capacitors. The electrode nodes e1-e32 for each of the lead-based electrodes are connected to bleed resistors/switches 922(1) -922(32), respectively. The bleed resistors typically have a high resistance, for example, about 5 MΩ. The bleed resistors/switches may each be individually controlled via a bleed enable signal <BL(en)(n)>, which when asserted, connect the respective electrode node to the bleed bus 908 through the bleed resistor. Notably, the bleed resistors for each of the electrode nodes may be individually selected and controlled. The residual charge that is bled to the bleed bus 908 may be bled to the case electrode by closing a switch 924. That DC path includes a resistor 926, which may be about 1 MΩ, for example. That DC path may be driven to V(ref) by closing a switch 928. Each of these switches may be controlled using appropriate control signals, which are omitted in the drawing for the sake of clarity. Note that when the switches 924 and 928 are both open, the bleed bus 908 simply bleeds the residual charge from the lead-based electrode nodes e1-e32 to the inside of the case electrode's blocking capacitor Cc. The case electrode node ec is also equipped with a bleed resistor/switch 930, which may also have a resistance of about 5 MΩ. The case bleed electrode may be controlled by a case bleed enable signal <BL(en)(c)>. Closing the case bleed resistor/switch 930 bleeds charge stored on Cc to the reference voltage V(ref). Likewise, closing switch 924 bleeds charge stored on Cc to the case electrode itself through the resistor 926 and to the tissue resistance.

It will be apparent to a person of skill in the art that the bleed switches/resistors and bleed network of the IPG circuitry 900 illustrated in FIG. 9 provides substantial flexibility in how residual charges stored on the various blocking capacitors CC-C(32) may be discharged. The various paths may provide different time constants for bleeding the accumulated residual charges. In the uses cases described below the discussion will focus on the timing of the independently controllable bleed enable signals that are configured to bleed the blocking capacitors for the various electrodes. The use cases will not specify which DC paths may be instantiated to accept the stored charges, i.e., whether the capacitors are bled to the case blocking capacitor Cc, the case electrode Ec itself, and/or the reference voltage V(ref). It is within the ability of a person of skill in the art, given the present disclosure, to make that decision.

FIGS. 10A and 10B illustrate a scenario wherein the IPG circuitry 900 (FIG. 9) is used to provide stimulation to a patient's tissue and to sense electrical potentials within the patient's tissue. Specifically, in the illustrated example, the electrodes E1 and E2 are configured to issue biphasic bipolar stimulation and the electrode E8 is configured to sense the electric potentials, as shown in FIG. 10A. One pulse of a stimulation wave form is illustrated and divided into time segments or “phases” comprising a pre-stimulation phase that occurs before the electrical stimulation is delivered, an active stimulation phase during which E1 and E2 are actively delivering stimulation, a passive charge recovery phase during which charges on the blocking capacitors C(1) and C(2) are being passively recovered, a sensing phase during which electrical potentials are being sensed at the electrode E8, and a quite phase that precedes the next pulse. Assume that the case electrode is configured to provide a common mode reference voltage V(ref) to the patient's tissue, as described above.

FIG. 10B shows the status of various components of the IPG circuitry (with reference to FIG. 9) during the various phases of the stimulation pulse. First consider what is happening with respect to the case electrode. Since the case electrode is being used to drive the tissue to a common mode reference voltage V(ref) as described above, the tissue drive switch for the case electrode 914(c) may be closed throughout all the phases of the stimulation waveform to provide the reference voltage to the tissue. In other words, the tissue drive enable signal TD(en)(c) may be asserted throughout the stimulation waveform. According to other embodiments, the tissue drive switch may be open during some phases when the reference voltage is not necessary.

Now referring to the electrodes E1 and E2, since the electrodes E1 and E2 are used to deliver the active stimulation, the PDAC and NDAC for each of those electrodes are active during the delivery of active stimulation and are inactive throughout the other phases of the stimulation waveform. Thus, those electrodes are instantiated to deliver the active stimulation during the active stimulation phase. In the pre-stimulation phase, prior to delivering the active stimulation, the bleed resistors/switches for E1 and E2 (922(1) and 922(2), respectively) may be closed to allow residual charge stored on each electrode's blocking capacitors C(1) and C(2) to be bled, as described above. Generally, any of the bleed paths described above may be used. During the active stimulation phase, the bleed resistors/switches 922(1) and 922(2) for the stimulating electrodes are open. Those bleed resistors/switches remain open during passive charge recovery, while the passive charge recovery resistors/switches for the electrodes E1 and E2 (916(1) and 916(2), respectively) are closed to instantiate passive charge recovery. Once passive charge recovery is completed at the electrodes E1 and E2, the passive charge recovery switches may be opened (ending passive charge recovery) and the bleed resistors/switches for E1 and E2 (922(1) and 922(2), respectively) may be closed to bleed residual charge from the blocking capacitors C(1) and C(2). The E1 and E2 bleed resistors/switches may remain closed throughout the sensing and quiet phases to continue to bleed charge from C(1) and C(2) and may then be opened in preparation for the next active pulse.

Now consider the circuitry with respect to the sensing electrode E8, which is configured for sensing electrical potentials in the patient's tissues. During the sensing phase of the waveform, the sense select switch 910(8) is closed to connect the electrode E8 to the sensing circuitry 912. Likewise, the bleed resistor/switch 922(8) for the sensing electrode E8 may be closed during all of the other phases when E8 is not actively being used for sensing to ensure that residual charge is bled from E8's blocking capacitor in preparation for sensing, thereby minimizing DC offset in the sensing measurement, as described above. Generally, the bleed resistor/switch 922(8) is open when E8 is actively sensing electric potentials at its electrode node.

A person of skill in the art will recognize that other timings of the various circuit elements described with respect to FIGS. 9, 10A, and 10B are possible and the timings described here may be modified to meet the requirements of particular stimulation and sensing criteria, as well as other functions executed by the IPG. For example, U.S. Patent Publication Nos. U.S. 2024/0058611 and 2023/0173273, the entire contents of which are each incorporated herein by reference, describe IPGs comprising offset compensation circuitry that is configured to issue compensating current to sensing electrode nodes, thereby charging/discharging the electrode nodes' DC-blocking capacitors to equate any DC-offset present at the electrode nodes' sensing amplifier circuitry. If the bleed resistors and such active DC-offset functions are instantiated at the same time, the bleed resistor circuitry and the active DC-offset functions may conflict. Accordingly, the bleed resistors may be individually controlled to open the relevant electrode nodes' bleed resistor switches while active DC-offset is occurring to prevent such conflicts.

It will also be recognized that the scenario illustrated above is quite simple. In more complex stimulation and sensing modalities, the ability to individually control the bleed resistors for each of the electrodes to prepare them for sensing is apparent. For example, since both the passive charge recovery network and the bleed switch/resistor network both “see” the inside plate of the DC-blocking capacitors for every electrode node, those two networks may be selectively connected together to selectively bleed charge from one electrode node's DC-blocking capacitor to the DC-blocking capacitors of any or all of the other nodes' DC-blocking capacitors.

A person of skill in the art will recognize that the individually controllable bleed resistors described herein provide flexibility in managing residual charges that may build up on various of the DC-blocking capacitors, which charges may confound the sensing of small amplitude potentials. In other words, the described system addresses the DC-offset problem described above. Generally, the DC-blocking capacitors of any of the electrode nodes may be bled at any time that the relevant electrode node is not being actively used for another purpose, such as providing stimulation, passive charge recovery, sensing, etc. The bleed resistors/switches of some of the electrode nodes may be selectively/controllably closed to bleed charge at those selected electrode nodes while the bleed resistors/switches of the non-selected electrode nodes are open, for example, because the non-selected electrode nodes are being used for a different purpose. In the illustrated example, the bleed resistors/switches of the sensing electrode node(s) are closed for bleeding charge at the sensing electrode nodes at the same time the bleed resistors/switches of the stimulating electrode nodes are open and the stimulating electrode nodes are providing active stimulation and passive charge recovery. The electrode nodes for the stimulating electrodes are then closed for charge bleeding once stimulation and passive charge recovery is completed and at the same time sensing is occurring at the sensing electrode nodes. This flexibility is possible because the bleed resistors/switches for each of the electrode nodes are all individually addressable/controllable.

In another example, assume that the same electrode nodes (e.g., e1 and e2) are selected both for providing active stimulation (and optionally passive charge recovery) and are also selected for sensing. In such an example, the bleed resistors/switches for e1 and e2 may be closed for bleeding charge from C1 and C2 before the active stimulation begins. Once active stimulation (and optionally passive charge recovery) begins, the bleed resistors/switches for e1 and e2 may be opened. Those bleed resistors/switches may be closed again to bleed charge before instantiating e1 and e2 for sensing and they may be opened again once sensing begins. Once sensing is completed, the bleed resistors/switches for e1 and e2 may be closed again to bleed residual charge from C1 and C2 in preparation for the next pulse.

It will be appreciated that the control circuitry of the IPGs described herein (e.g., μC 102, FIG. 5), may be configured to provide the enable signals <BL(en)(n)> for controlling the bleed resistor/switches, as well as all of the enable/control signals mentioned herein. The IPGs' control circuitry may be programmable, for example, using any of the external devices described above.

Although particular embodiments of the present invention have been shown and described, the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A stimulator device configured to provide electrical stimulation, comprising:
a plurality of electrode nodes, wherein each electrode node is couplable to a different electrode, wherein each electrode is configured to contact a patient's tissue,
driver circuitry configured to drive at least two of the electrode nodes to provide current through the tissue,
a plurality of DC-blocking capacitors, wherein each of the DC-blocking capacitors is connected in series between one of the electrode nodes and a different one of the electrodes,
a plurality of first switches and a plurality of first resistors, wherein each of the plurality of first switches is serially connected to a different one of the plurality of first resistors, wherein each of the serially connected first switch and first resistor is coupled at one end to a different one of the plurality of electrode nodes and at a second end to a first bus,
a plurality of second switches and a plurality of second resistors, wherein each of the plurality of second switches is serially connected to a different one of the plurality of second resistors, wherein each of the serially connected second switch and second resistor is individually controllable and is coupled at one end to a different one of the plurality of electrode nodes and at a second end to a second bus,
control circuitry configured to:
close at least one selected first switch to discharge charge on at least one of the DC-blocking capacitors to the first bus, and
close at least one selected second switch to discharge charge on at least one of the DC-blocking capacitors to the second bus.

2. The stimulator device of claim 1, wherein second bus comprises a third resistor in parallel with one of the DC-blocking capacitors and wherein the control circuitry is configured to selectively close a third switch to enable charge on the second bus to discharge through the third resistor.

3. The stimulator device of claim 2, wherein the stimulator device comprises a case that is configurable as a case electrode, wherein the third resistor is in parallel with the DC-blocking capacitor of the case electrode, and wherein charge discharged through the third resistor is discharged to the tissue via the case electrode.

4. The stimulator device of claim 1, comprising a plurality of fourth switches wherein each of fourth switches is individually coupled at one end to a different one of the plurality of electrode nodes and at a second end to a third bus, wherein the third bus is connected to a reference voltage source that is configured to provide a reference voltage.

5. The stimulator device of claim 4, wherein the control circuitry is configured to close one or more of the fourth plurality of switches to provide the reference voltage to a selected one or more of the electrode nodes.

6. The stimulator device of claim 4, wherein the driver circuitry is powered by a compliance voltage (VH) and wherein the reference voltage is a function of VH.

7. The stimulator device of claim 6, wherein the reference voltage is VH/2.

8. The stimulator device of claim 4, comprising a battery, wherein the reference voltage is a battery voltage or ground.

9. The stimulator device of 4, wherein the control circuitry is configured to selectively connect the second bus to the third bus.

10. The stimulator device of claim 1, comprising a plurality of fifth switches, wherein each of fifth switches is individually coupled at one end to a different one of the plurality of electrode nodes and at a second end to a fourth bus, wherein the fourth bus is connected to sensing circuitry.

11. The stimulator device of claim 10, wherein the control circuitry is configured to:
select one or more of the plurality of electrode nodes as sensing electrode nodes,
during a first phase, close the second switch of each of the sensing electrode nodes to bleed residual charge from the sensing electrode node's DC-blocking capacitor to the second bus,
during a second phase, open the second switch of each of the sensing electrode nodes and close the fifth switch of each of the sensing electrode nodes.

12. The stimulator device of claim 11, wherein the sensing circuitry comprises a plurality of sensing amplifiers and wherein closing the fifth switch of each of the sensing electrode nodes comprises asserting sensing enable signals to each of the fifth switches, wherein the each of the sensing enable signals control the respective fifth switches to connect each of the sensing electrode nodes to selected sensing amplifiers of the plurality of sensing amplifiers.

13. The stimulator device of claim 1, wherein the control circuitry is configured to:
select at least two of the electrode nodes as stimulating electrode nodes,
during a first phase, cause the driver circuitry to drive the selected at least two stimulating electrode nodes to provide current through the tissue,
during a second phase, use passive charge recovery to recover at least a portion of charge from the DC-blocking capacitors connected to the at least two stimulating electrode nodes, and
during a third phase, bleeding residual charge from the DC-blocking capacitors connected to the at least two stimulating electrode nodes, wherein the residual charge comprises charge on the DC-blocking capacitors that was not recovered during the second phase.

14. The stimulator device of claim 13, wherein the control circuitry is configured to:
open the first and second switches connected to the at least two stimulating electrode nodes during the first phase,
close the first switches connected to the at least two stimulating electrode nodes during the second phase, and
open the first switches and close the second switches connected to the at least two stimulating electrode nodes during the third phase.

15. The stimulator device of claim 14, wherein during the third phase, the control circuitry is configured to close a third switch, causing the residual charge to bleed to a selected electrode through a third resistor that is parallel across the selected electrode's DC-blocking capacitor.

16. The stimulator device of claim 15, wherein the selected electrode is a case electrode.

17. A stimulator device configured to provide electrical stimulation, comprising:
a plurality of electrode nodes, wherein each electrode node is couplable to a different electrode configured to contact a patient's tissue, wherein each electrode node may be configured as a stimulating electrode node to provide active stimulation or as sensing electrode node to sense electric potentials present at the electrode node,
a plurality of DC-blocking capacitors, wherein each of the DC-blocking capacitors is connected in series between one of the electrode nodes and a different one of the electrodes,
a plurality of passive charge recovery resistors, wherein each of the passive charge recovery resistors is coupled to a different one of the plurality of electrode nodes to passively recover charge from the node's DC-blocking capacitor,
a plurality of individually controllable bleed resistors, wherein each of the bleed resistors is connected to a different one of the plurality of electrode nodes to bleed residual charge from the node's DC-blocking capacitor, and
control circuitry configured to:
selectively activate a first one or more of the bleed resistors and not activate a second one or more of the bleed resistors.

18. The stimulation device of claim 17, wherein the control circuitry is configured to:
select a two or more of the plurality of electrode nodes as stimulating electrode nodes,
select one or more of the plurality of electrode nodes as sensing electrode nodes,
during a first phase, cause the two or more stimulating electrode nodes to deliver active stimulation,
during a second phase, use the passive charge recovery resistors for the stimulating electrode nodes to passively recover charge from the stimulating electrode nodes' DC-blocking capacitors,
during a third phase, use the one or more sensing electrode nodes to sense electric potentials present at the one or more sensing electrode nodes,
during one or more of the first and second phases, activate the sensing electrode nodes' bleed resistors to bleed residual charge from the sensing electrode nodes' DC-blocking capacitors while not activating the stimulating nodes' bleed resistors.

19. The stimulation device of claim 18, wherein the control circuitry is configured to:
during the third phase, activating the stimulating nodes' bleed resistors to bleed residual charge from the stimulating nodes' DC-blocking capacitors while not activating the sensing nodes' bleed resistors.

20. The stimulation device of claim 17, wherein each of the plurality of passive charge recovery resistors are variable resistors and wherein each of the plurality of bleed resistors are not variable resistors.
